(19) United States
(12) Patent Application Publication
(10) Pub. No.: US 2011/0184053 A1
(43) Pub. Date: Jul. 28, 2011

(54) NOVEL NUCLEIC ACID TRANSFER SYSTEM

(75) Inventors: Patrick Couvreur, Villebon Sur Yvette (FR); Didier Desmaele, Fresnes (FR); Mouna Raouane, Paris (FR); Liliane Massade, Cachan (FR); Claude Malvy, Boussy Saint Antoine (FR)

(73) Assignees: CENTRE NATIONAL DE LA RECHERCHE SCIENTIFIQUE, PARIS (FR); UNIVERSITE PARIS SUD, ORSAY (FR)

(21) Appl. No.: 12/992,872

(22) PCT Filed: May 14, 2009
(86) PCT No.: PCT/FR09/50887

§ 371 Date: Mar. 24, 2011

(30) Foreign Application Priority Data
May 16, 2008 (FR) 0853205

(51) Int. Cl. A61K 31/7088; C07H 21/00; C07H 21/02; A61P 35/00; B82Y 5/00; B82Y 40/00

(52) U.S. Cl. 514/44 R; 536/23.1; 536/24.5; 977/773; 977/795; 977/840

(57) ABSTRACT

The invention relates to a complex formed by at least one molecule of nucleic acid comprising between 10 and 40 nucleotides, covalently coupled to at least one hydrocarbon compound that is at least C₁₈ is hydrocarbon compound, having a squalene structure or a structure similar thereto.

The present invention relates to the field of molecular biology, and in particular aims to propose a new type of vehicle that is of use in gene therapy, for nucleic acid vectorization.

Gene therapy is based mainly on the use of nucleic acids for therapeutic purposes, thus contributing to the obtaining of “medicament DMA”. Initially limited to the context of hereditary genetic diseases, where a functional copy of the causal gene is introduced into the somatic cells affected by the hereditary defect, gene transfer has been extended to the treatment of acquired diseases, such as cancer, for example.

More recently, gene therapy has been extended to the transfer of short nucleic acid sequences, with the aim either of modifying the expression of a gene (exon skipping, for example), or of repairing, by genetic recombination, the causal genetic abnormality (short sequences of DNA, DNA/RNA chimeras, for example), or else of inhibiting the expression of a gene (siRNA or “Small Interfering RNA”, antisense oligonucleotides, for example).

The inventors of the present invention have more particularly focused on the latter type of therapeutic strategy.

RNA interference is a phenomenon that has been highly conserved during evolution. It is based on a mechanism using small, generally double-stranded, RNAs, called siRNAs, generally comprising from 21 to 25 nucleotides, which, once transfected into the cell, block the translation of mRNAs (messenger RNAs) by sequence complementarity with said mRNAs.

These siRNAs can be transfected as such into the cell or can be derived from the degradation of a larger RNA molecule.

In the latter case, the double-stranded RNAs present in a cell are first of all processed by a type III ribonuclease called Dicer. The latter cleaves the double-stranded RNA every 21 to 25 base pairs, thus forming siRNAs. Dicer then transfers the siRNAs to a multiprotein complex, the RISC complex (RNA-Induced Silencing Complex). One of the strands of the si RNA, called “passenger”, is eliminated, while the other, called “guide”, directs the RISC complex to the mRNAs having a sequence complementary to the guide strand. If the complementarity between the siRNA and the target mRNA is perfect, the RISC complex cleaves the target mRNA, which is then degraded and is therefore no longer translated into protein.

Thus, siRNAs transfected as they are or derived from the cleavage of longer RNA result in the silencing of target gene expression. A few noncomplementary bases are sufficient to prevent the cleavage. This mechanism is therefore very specific for the siRNA sequence and for its target, the mRNA.

However, many genes are overexpressed or expressed at the wrong place or at the wrong time in many pathological conditions. The possibility of being able to inhibit these pathological expressions is a great hope for treating these many diseases, at the forefront of which are cancers, but also diseases of viral or autoimmune origin.

The use of siRNAs represents an advantageous tool for studying the function of certain genes. This is because, by blocking the expression of a target gene, it is possible to determine its function, by default.

However, the use of this therapeutic strategy in vivo raises even more difficulties, in particular in terms of implementation. This is because nucleic acid transfection in the context of gene therapy mainly comes up against three obstacles.

First of all, in order to be effective, nucleic acids must necessarily reach the target cells specifically targeted and cross the cell membranes, while at the same time not being degraded by the organism. Next, the nucleic acid transfection must also be acceptable in terms of toxicity. Finally, nucleic acid molecules have poor stability in vivo.

In order to overcome these difficulties, several vectorization techniques have already been proposed for the transfer of nucleic acid molecules into cells.

A first technique uses the adenovirus (or AAV, for Adeno-Associated Virus), which is a recombinant virus. Another mode of transfection, electrotransfer, consists in applying an electric field to the cells under consideration, after having injected a nucleic acid molecule into said cells. Specifically, although these first two methods make it possible to obtain satisfactory gene transfer levels, they have, on the other hand, a drawback in terms of toxicity.

Another technique which in fact makes it possible to do away with this drawback takes advantage of synthetic vectors. Examples of synthetic vectors generally used for nucleic acid transfection are, for example, lipoplexes which are complexes of nucleic acids and of cationic lipids, optionally in combination with neutral lipids. These vectors, which are positively charged, are essentially internalized in the cell by means of nonspecific electrostatic interactions. By way of example, chitosan, which is a positively charged polysaccharide containing nonacetylated β-D-glucosamine residues, has been widely studied for its use in nucleic acid delivery systems. Thus, siRNA delivery systems in which the siRNA is adsorbed at the surface of the “core/crown” nanoparticles, the core comprising poly(isobutyl cyanoacrylate) and the crown comprising chitosan, are known from H. de Martimprey et al., Nucleic Acids Res. 2008; 36(1).

However, the implementation of a delivery system using lipid cations generally requires a great deal of perfecting before use in vivo in order to hope for optimum transfection efficiency.

Furthermore, the formation of the nucleic acid/synthetic vector complex is not always immediate and can be restrictive in terms of synthesis. In addition, compared with viral vectors, synthetic vectors remain less efficient in vivo for targeting a tissue after intravenous injection. Finally, all these systems have, by virtue of their cationic nature, a natural tendency to interact strongly with the negatively charged plasma proteins, which can lead to aggregation phenomena and toxic reactions.

The object of the present invention is in fact to propose a novel mode of synthetic vectorization, which is noncationic in nature, for nucleic acid transfection, which makes it possible to respond to the above mentioned drawbacks.

In particular, the present invention results from the unexpected observation by the inventors that the coupling of a molecule of nucleic acid comprising from 10 to 40 nucleotides to an at least ds hydrocarbon-based compound of squalenoyl radical type, or an analog thereof, results in the formation of nanoparticles, constituting a delivery system efficient in terms of vectorization and transfection, and which in addition is acceptable in terms of toxicity.

WO 2006/090029 already describes the ability of squalene, when it is covalently coupled to gemcitabine, which is a hydrophilic and polar molecule, to spontaneously form nanoparticles of about a hundred nanometers in an aqueous medium. This capacity is in particular explained therein by the amphiphilic behavior of the derivatives thus synthesized, the squalene part representing the hydrophobic part and the gemcitabine part representing the hydrophilic part.

Against all expectations, the inventors have noted that this ability of squalene and of analogs thereof to form nanoparticles can also manifest itself when said squalene is covalently associated with a small nucleic acid molecule, such as an siRNA or an antisense oligonucleotide, with, however, a molecular weight which remains much higher than that of the nucleoside analogs considered in WO 2006/090029. Unexpectedly, the nanoparticles thus formed are found to also be of sufficiently small size (from a few tens of nanometers to a few hundred nanometers) to be compatible with an application in gene therapy, via the systemic route.

Thus, according to a first aspect, the present Invention relates to a complex formed by at least one molecule of nucleic acid comprising from 10 to 40 nucleotides, covalently coupled to at least one hydrocarbon-based compound that is at least a C₁₈ hydrocarbon-based compound, of squalene structure or an analog thereof.

According to one preferred embodiment of the present invention, the molecule of nucleic acid comprising from 10 to 40 nucleotides is an antisense oligonucleotide or, most particularly preferably, an RNA molecule, and in particular an siRNA molecule.

Thus, a subject of the invention is directed toward a complex in accordance with the invention in which the nucleic acid molecule is an RNA molecule, and in particular an siRNA molecule.

Other nucleic acid molecules are also capable of being vehicled by the vectorization system according to the invention, such as aptamers.

In this respect, it is most particularly possible to use sequences that are of use in the treatment of cancers, such as RET/PTC1 siRNA and RET/PTC3 siRNA (papillary thyroid carcinoma), EWS-flil siRNA (Ewing's sarcoma), anti-ICAM antisense oligonucleotide and anti-ICAM siRNA (Kretschmer-Kazemi Far, R. et al. Nucl. Acids Res. 2003 31:4417-4424), anti-RhoA and anti-RhoC siRNAs (Pille et al., (2005). Anti-RhoA and anti-RhoC siRNAs inhibit the proliferation and invasiveness of MDA-MB-231 breast cancer cells in vitro and in vivo. Mol Ther 11, 267-574), anti-K-ras siRNAs (Martin S E, et al. Multiplexing siRNAs to compress RNAi-based screen size in human cells. Nucleic Acids Res. 2007; 35(8) and the siRNA which inhibits the expression of growth factors such as VEGF-A and VEGF-C (for Vascular Endothelial Growth Factor type A or B) (Filleur et al., Cancer Res., 2003.

In particular, the RET/PTC1 siRNA is an siRNA which has a sequence complementary to the chimeric oncogene RET/PTC1 (for Papillary Thyroid Carcinoma), resulting from a chromosomal rearrangement and in particular from fusion of the tyrosine kinase domain of she RET protooncogene (membrane receptor having tyrosine kinase activity) with the 5′ end of another gene, in the case in point H4. This chimeric oncogene is detected in particular in an individual suffering from papillary thyroid carcinoma.

Several RET/PTC1 siRNAs have been screened, selected and cloned (H. De Martimprey et al., 2007, cf. below).

The RET/PTC1 siRNA sequence is more particularly considered according to the invention.

A subject of the invention is therefore directed toward a complex according to the invention, in which the nucleic acid molecule is a RET/PTC1 siRNA having the sequence which follows: 5′-CGUUACCAUCGAGGAUCCAdAdA-3′ (SEQ ID No: 1).

According to another subject of the invention, the two entities forming the complex according to the present invention are coupled by means of a covalent bond of ester, ether, thioether, disulfide, phosphate or amide type, preferably disulfide type.

According to one particular embodiment, a complex is formed by at least one molecule of nucleic acid comprising from 10 to 40 nucleotides, covalently coupled to at least two hydrocarbon-based compounds that are at least C₁₈ is hydrocarbon-based compounds, of squalene structure, or an analog thereof.

Another subject of the present invention is directed toward the nanoparticles of a complex according to the present invention, as described above.

According to another subject, the present invention is directed coward a method for preparing nanoparticles according to the present invention, characterized in that it comprises at least:
the dispersion of a complex according to the invention in at least one organic solvent, for example an alcohol such as ethanol, at a concentration that is sufficient to obtain, when the resulting mixture is added, with stirring, and generally dropwise, to an aqueous phase, the instantaneous formation of nanoparticles of said complex in suspension in said aqueous phase, and
where appropriate, the isolation of said nanoparticles.

Particularly preferably, the siRNA used in a complex according to the invention and in the nanoparticles formed from this complex is the RET/PTC1 siRNA (SEQ ID No.: 1).

According to another of its subjects, the present invention is directed toward a complex and/or the corresponding nanoparticles according to the invention, for the use thereof in the treatment and/or prevention of cancers, in particular endocrine cancers, and more particularly papillary thyroid carcinoma, or else in the treatment and/or prevention of viral pathological conditions.

The invention is also directed toward pharmaceutical compositions comprising at least one complex and/or nanoparticles according to the invention, in combination with at least one acceptable pharmaceutical vehicle.

### Hydrocarbon-Based Compound of Squalene Structure

For the purpose of the present invention, a “compound of squalene structure” is a compound comprising at least one squalenoyl radical, in particular as defined hereinafter.

For the purpose of the present invention, a “squalenoyl radical” is capable of reproducing the self-organizing properties manifested by squalene and/or derivatives thereof when it is placed in contact with an aqueous medium.

In particular, a “squalenoyl radical” according to the invention can be represented schematically by the linear hydrocarbon-based structure formed by isoprene units, which can be represented by formula (I) which follows:

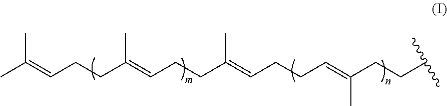

in which:
m=1, 2, 3, 4 or 5;
n=0, 1, 2, 3, 4 or 4; and

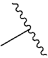

represents the bond to the rest of the complex with the nucleic acid.

According to one preferred embodiment of the present invention, use is made of a squalenoyl radical of formula (I′) which follows, corresponding to a radical of preceding formula (I) in which n=0:

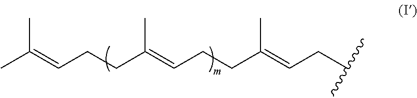

More particularly, a squalenoyl radical of formula (I) according to the invention, for which m=1 and n=3, can have the structure of squalene or of derivatives thereof, having the following formula:

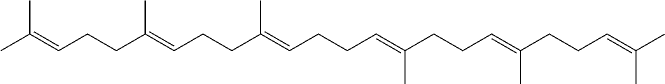

According to one preferred embodiment of the invention, use is made of a radical of formula (I) in which m=1 and n=2.

Thus, a subject of the invention relates to a complex in accordance with the present invention, in which the hydrocarbon-based compound that is at least a C₁₈ hydrocarbon-based compound, of squalene structure or an analog thereof is represented by a radical of formula (I) as defined above.

By way of illustration of these hydrocarbon-based compounds, mention may more particularly be made of squalenic acid and derivatives thereof, such as 1,1′,2-trisnorsqualenic acid. Mention may also be made, by way of example, of squalene (also called spiracene or sirprene), isoprenoid containing 30 carbon atoms and 50 hydrogen atoms (chemical name: (E)-2,5,10,15,19,23-hexamethyl-2,6,10,14,18,22-tetracosahexene). Squalene is an essential intermediate of cholesterol biosynthesis.

A subject of the invention is also directed coward complexes in accordance with the present invention containing at least one of the particular hydrocarbon-based compounds mentioned above.

In particular, a subject of the present invention is directed toward a complex according to the invention, in which the hydrocarbon-based compound that is at least a C₁₈ hydrocarbon-based compound, of squalene structure, is 1,1′,2-trisnorsqualenic acid.

As the inventors have noted, this squalenoyl radical is particularly important in the context of the present invention since it spontaneously displays, when it is placed in contact with a polar medium, and more particularly water, a compacted conformation.

Unexpectedly, the inventors have noted that this capacity remains when such a radical is combined with and in particular covalently bonded to a molecule of nucleic acid comprising from 10 to 40 nucleotides. This results in the generation of a compacted architecture in the form of nanoparticles, in which the two nucleic acid/compound of squalene structure entities are intimately interlaced in each other.

For the purpose of the present invention, the term “analog” denotes a hydrocarbon-based compound that is capable, on the one hand, of reproducing the behavior of a compound of squalene structure when it is placed in contact with a polar medium, and that is capable, on the other hand, of reproducing this capacity when it is bonded to a nucleic acid molecule according to the invention. The substituted forms of squalene derivatives, and in particular squalenic acid and its derivatives, especially of substitution, are especially covered by this definition. By way of example of a squalenic acid derivative, mention may be made of 1,1′,2-trisnorsqualenic acid.

In general, at least one hydrocarbon-based compound of squalene structure is covalently bonded to a nucleic acid molecule. Of course, the number of molecules of hydrocarbon-based derivative capable of interacting with a nucleic acid molecule may be greater than 1.

### Nucleic Acid/Compound of Squalene Structure Complex

The formation of the nucleic acid/compound of squalene structure complex requires that the two entities of the complex bear functions capable of forming a covalent bond or a linker arm, as described above.

The hydrocarbon-based compound of squalene structure generally bears a function capable of reacting with a function present on the nucleic acid molecule under consideration so as to establish a covalent linkage between the two entities, for example of ester, ether, thioether, disulfide, phosphate or amide type, thus forming a covalent complex. Advantageously, it is a thiol function. In this case, the hydrocarbon-based compound of squalene structure is 1,1′,2-trisnorsqualenic acid or a derivative thereof, and in particular an amide or ester thereof.

According to one variant embodiment, the covalent linkage that exists between the two types of molecules can be represented by a spacer or alternatively a linker arm. Such an arm may in particular prove to be useful for increasing the force of the nucleic acid/squalenoyl radical interaction.

Such an arm in fact makes if possible to introduce, via each of the two ends of its backbone, the appropriate functions, i.e. functions respectively having the expected reactional affinity, one for the function present on the derivative of squalene structure and the other for the function present on the nucleic acid molecule under consideration.

It may also be envisioned that this linker arm also has in its backbone a labile function, which is subsequently suitable for separating the compound of squalene structure from the nucleic acid molecule under consideration. It may, for example, be a peptide unit that can be recognized by an enzyme.

Units of linker arm type are well known to those skilled in the art and their use clearly falls within the competence thereof.

By way of representation of the linker arms that may be envisioned according to the invention, mention may in particular be made of (poly)amino acid units, polyol units, saccharide units, and polyethylene glycol (polyetheroxide) units of low molecular weight.

Thus, for the purpose of the present invention, a “covalent linkage” preferably represents a covalent bond, in particular as specified above, but also covers a covalent linkage represented by a linker arm as defined above.

Thus, the covalent complex according to the present invention can be represented by the compound of formula (II) which follows:

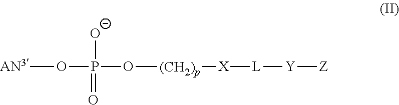

AN represents a molecule of nucleic acid comprising from 10 to 40 nucleotides, the 3′ end of which is linked to the rest of the complex,
X represents a covalent linkage between the two entities, and more particularly a linkage of ester, ether, thioether, disulfide, phosphate or amide type,
L represents a linker arm, preferably chosen from saturated alkyl chain units, or else (poly)amino acid units, polyol units, saccharide units and polyethylene glycol (polyetheroxide) units of low molecular weight, namely a molecular weight ranging from 40 to 500 daltons,
Y represents a covalent linkage, and more particularly a linkage of ester, amide, thioether or phosphate type,
p represents 0, 1, 2, 3 or 4,
Z represents a squalenoyl radical or derivative thereof, of formula (I) or (I′) above.

For the purpose of the present invention:
the term “saturated alkyl chain” is intended to mean a linear or branched, saturated alkyl radical which can contain from 1 to 8 carbon atoms, in which two bonds to a hydrogen atom are replaced wish two covalent bonds to the rest of the molecule,
the term “saccharide unit” is intended to mean a radical comprising at least one radical chosen from trioses (glyceraldehyde, dihydroxyacetone), tetroses (erythrose, threose, erythrulose), pentoses (arabinose, lyxose, ribose, deoxyribose, xylose, ribulose, xylulose), hexoses (allose, altrose, galactose, glucose, gulose, idose, mannose, talose, fructose, psicose, sorbose, tagatose), heptoses (mannoheptulose, sedoheptulose), octoses (octolose, 2-keto-3-deoxymannooctonate), isonoses (sialose), and
   the term “(poly) amino acid unit” is intended to mean a unit having at least one unit:

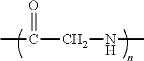

in which n is greater than or equal to 1.

The present invention relates more particularly to a complex, represented by the compound of formula (IIA) which follows:

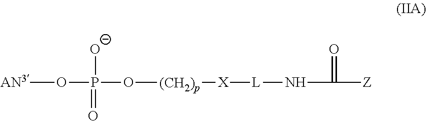

in which:
AH, X, L, Z and p have the same definitions as for the compound of formula (II).

In the examples of formulae (II) and (IIA), L preferably represents a saturated alkyl chain as defined above.

In the complexes of formulae (II) and (IIA), AN is preferably a molecule of ribonucleic acid, preferably comprising from 19 to 25 nucleotides.

Particularly preferably, the ribonucleic acid is an siRNA, in particular a RET/PTC1 siRNA of sequence corresponding to SEQ ID No.: 1.

In particular, a subject of the present invention is directed toward a complex of formula (II) in which X is a disulfide covalent linkage and Z represents a radical of formula (I) in which m represents 1 and n represents 2.

The reaction necessary for the establishment of at least one covalent bond between at least one molecule of nucleic acid under consideration and at least one compound of squalene structure or analog thereof can be carried out according to standard conditions, and the implementation thereof is therefore clearly part of the knowledge of those skilled in the art.

This reaction is generally carried out in solution in the presence and with an excess of at least one compound of squalene structure relative to the nucleic acid molecule under consideration, for example in a proportion of two equivalents, according to the standard conditions required for interaction between the two specific functions borne by each of the two entities.

Prior to this reaction, each of the two entities, on the one hand, the nucleic acid molecule and, on the other hand, the hydrocarbon-based compound, are modified in order to bear a function capable of establishing a covalent linkage between them. Preferably, each of the two molecules carries a thiol function in order to establish a disulfide bridge between them.

The functionalization of an RNA molecule is widely documented in the prior art. For example, it can be functionalized with a thiol function, by analogy with the method described in Gnaccarmi et al., J. Am. Chem. Soc. 2006, 128, 8063-8067, i.e. by means of an appropriate phosphoramidite. In the present case, this type of functionalization is grafted at the 3′ end of the siRNA molecule.

For its part, the compound of squalene structure is functionalized, in particular with a thiol function, by analogy with the method described in Elbright et al., Biochemistry 1992, 31, 10664-10670, i.e. using a pyridyl disulfide derivative.

The conjugation of the two partners thus functionalized is carried out by analogy with the coupling method described in Sengle et al. Biorg. & Med. Chem., 2000, 8, 1317-1329, also known as “GMPS-coupling method”.

A subject of the present invention is therefore also directed toward a complex in accordance with the invention, represented by formula (II) or (IIA) as defined above.

### Nanoparticles According to the Invention

As specified previously, the covalent coupling of at least one nucleic acid molecule under consideration according to the invention with at least one molecule of a hydrocarbon-based compound of squalene structure is of a nature to give the nucleic acid thus complexed an ability to become organized in a compacted form in a polar solvent medium, thus leading to the formation of nanoparticles.

In general, the nanoparticles thus obtained have a mean size ranging from 30 to 500 nm, in particular from 50 to 250 nm, or even from 100 to 400 nm, measured by light scattering using a Coulter® N4MD nanosizer from Coulter Electronics, Hialeah, USA.

A subject of the invention is directed toward nanoparticles in accordance with the invention, the mean size of which ranges from 30 to 500 nm, in particular from 50 to 250 nm, or even from 100 to 400 nm.

Thus, the interaction of a highly water-soluble nucleic acid molecule under consideration according to the invention with a squalenonyl derivative or an analog thereof, for instance 1,1′,2-trisnorsqualenic acid, gives said nucleic acid molecule physicochemical characteristics that are sufficient to impart thereon an ability to form particles of which the size proves to be compatible for parenteral administration, and in particular intravenous administration.

As indicated above, the nanoparticles according to the present invention can be obtained by means of a step of dispersion of a complex according to the invention, i.e. formed preliminarily by coupling of at least one compound of squalene structure or analog thereof to at least one nucleic acid molecule, according to the Invention, in at least one organic solvent, for example an alcohol such as ethanol, at a concentration that is sufficient to obtain, when the resulting mixture is added, with stirring, and generally dropwise, to an aqueous phase, the instantaneous formation of nanoparticles of said derivative in suspension in said aqueous phase, followed, where appropriate, by the isolation of said nanoparticles.

The reaction can generally be carried out at ambient temperature. Irrespective of its value, the reaction temperature should not affect the activity of the nucleic acid molecule under consideration. The method for preparing the nanoparticles according to the invention is particularly advantageous since it does not require the obligatory presence of surfactants.

According to one particular embodiment, the nanoparticles are obtained in the form of an aqueous suspension.

This property is particularly beneficial since a large number of surfactants do not prove to be compatible with an in vivo application.

However, it is understood that the use of surfactants, generally advantageously free of any toxicity, can be envisioned in the context of the invention. Surfactants of this type may, moreover, make it possible to obtain even smaller sizes during the formation of nanoparticles. By way of nonlimiting illustration of surfactants of this type which can be used in the present invention, mention may in particular be made of polyoxyethylene-polyoxypropylene copolymers, phospholipid derivatives and lipophilic derivatives of polyethylene glycol.

As a lipophilic derivative of polyethylene glycol, mention may, for example, be made of polyethylene glycol cholesterol. As examples of polyoxyethylene-polyoxypropylene block copolymers, mention may particularly be made of polyoxyethylene-polyoxypropylene-polyoxyethylene triblock copolymers, also known as Poloxamers®, Pluronics® or synperonics, and which are sold in particular by the company BASF.

Poloxamines, which are related to these families of copolymers, and which are constituted of hydrophobic segments (based on polyoxypropylene), hydrophilic segments (based on polyoxyethylene) and on a central portion deriving from the ethylenediamine unit, can also be used.

The nanoparticles according to the invention are of course capable of bearing, at the surface, a multitude of reactive functions, such as hydroxyl or amine functions, for example. It is therefore possible to envision attaching all sorts of molecules to these functions, in particular via covalent bonds.

By way of nonlimiting illustration of molecules of this type which are capable of being combined with the nanoparticles, mention may in particular be made of molecules of label type, compounds capable of performing a targeting function, and also any compound that is capable of imparting particular pharmacokinetic characteristics thereto. As regards this last aspect, it may thus be envisioned to attach to the surface of these nanoparticles lipophilic derivatives of polyethylene glycol, for instance the polyethylene glycol/cholesterol conjugate, polyethylene glycol phosphatidylethanolamine, or better still polyethylene glycol/squalene. Specifically, given the natural affinity of squalene residues for one another, the polyethylene glycol/squalene conjugate associates, in the case in point, with the nanoparticles according to the invention, and thus results in the formation of nanoparticles surface-coated with polyethylene glycol. Moreover, and as mentioned above, the polyethylene glycol/squalene conjugate advantageously acts, during the process of formation of the nanoparticles according to the invention, as a surfactant owing to its amphiphilic behavior and therefore stabilizes the colloidal solution, thus reducing the size of the nanoparticles formed. A surface coating based on such compounds, and in particular polyethylene glycol or the polyethylene glycol/cholesterol conjugate or the polyethylene glycol/squalene conjugate is in fact advantageous for imparting increased vascular remanence owing to a significant reduction in uptake of the nanoparticles by liver macrophages.

According to one advantageous embodiment, the nanoparticles according to the invention are formulated in the form of an aqueous dispersion with a view to their administration generally systemically.

According to another advantageous embodiment, this aqueous dispersion contains less than 5% by weight, or even less than 2% by weight, and more particularly is devoid of surfactant or the like, for instance polyethylene glycols and polyglycerol and their derivatives, such as the esters, for example.

According to another advantageous embodiment, this aqueous dispersion contains less than 5% by weight, or even less than 2% by weight of C₂ to C₄ alcohol, for instance ethanol.

According to another advantageous embodiment, this aqueous dispersion intrinsically has a viscosity compatible with intravenous administration.

Thus, the formulation, in an aqueous medium, of the nucleic acid under consideration by means of squalenic acid in the form of water-dispersible nanoparticles advantageously makes it possible to obtain a suspension of nanoparticles without any additive other than the 5% dextrose necessary to make the injectable suspension isotonic.

As indicated above, the present invention is also directed toward the use of at least one nanoparticle according to the invention in pharmaceutical compositions.

Another aspect of the invention therefore relates to a pharmaceutical composition comprising, as active material, at least one complex in accordance with the present invention, in particular in the form of nanoparticles. The complexes in accordance with the present invention may be combined therein with at least one pharmaceutically acceptable vehicle.

By way of examples of pharmaceutical formulations compatible with the compositions according to the invention, mention may in particular be made of:
intravenous injections or infusions;
saline solutions or solutions of purified water;
compositions for inhalation;
capsules, sugar-coated tablets, cachets and syrups in particular incorporating, as vehicle, water, calcium phosphate, sugars, such as lactose, dextrose or mannitol, talc, stearic acid, starch, sodium bicarbonate and/or gelatin.

When the complexes and/or nanoparticles are used as a dispersion in an aqueous solution, they may be combined with excipients such as sequestrants or chelating agents, antioxidants, pH regulators and/or buffering agents.

In addition to the abovementioned compounds, the pharmaceutical compositions according to the invention may contain agents such as preservatives, wetting agents, solubilizing agents and colorants.

Such compositions according to the invention, from the viewpoint of the nature of the nucleic acid under consideration in the form of nanoparticles according to the invention, can prove to be particularly useful in the treatment and/or prevention of cancers in mammals. Mention may be made, for example, without however being limited thereto, of carcinomas, sarcomas, hematopoietic cancers, breast cancer, colon cancer, rectal cancer, pancreatic cancer, testicular cancer, uterine cancer, cancer of the gastrointestinal tract, lung cancer, ovarian cancer, prostate cancer, mouth cancer, brain cancer, head cancer, neck cancer, throat cancer, kidney cancer, bone cancer, liver cancer, cancer of the spleen, bladder cancer, skin cancer, cancer of the larynx, cancer of the nasal passages, AIDS-related cancers, endocrine cancers, multiple myeloma (or bone marrow cancer), leukemias, Kaposi's sarcoma, and lymphomas, such as Hodgkin's disease or non-Hodgkins lymphoma.

For the purpose of the invention, the term “treatment” is intended to mean an inhibition of or a reduction in the degree of proliferation of cancer cells, enabling partial or complete remission.

Thus, in the case where the nucleic acid is the RET/PTC1 siRNA (SEQ ID No.: 1), such compositions are of use in the treatment and/or prevention of endocrine cancers such as thyroid cancer, and more particularly papillary thyroid carcinoma.

Thus, the complexes and/or nanoparticles according to the present invention are of use for the preparation of a pharmaceutical composition intended for the treatment and/or prevention of cancers, such as carcinomas, sarcomas, hematopoietic cancers, breast cancer, colon cancer, rectal cancer, pancreatic cancer, testicular cancer, uterine cancer, cancer of the gastrointestinal tract, lung cancer, ovarian cancer, prostate cancer, mouth cancer, brain cancer, head cancer, neck cancer, throat cancer, kidney cancer, bone cancer, liver cancer, cancer of the spleen, bladder cancer, skin cancer, cancer of the larynx, cancer of the nasal passages, AIDS-related cancers, endocrine cancers, multiple myeloma (or bone marrow cancer), leukemias, Kaposi's sarcoma, or lymphomas, such as Hodgkin's disease or non-Hodgkins lymphoma.

In particular, the complexes and/or nanoparticles according to the present invention are of use for the preparation of a pharmaceutical composition intended for the treatment and/or prevention of cancers, such as endocrine cancers, and more particularly papillary thyroid carcinoma.

The complexes or nanoparticles in accordance with the present invention can be administered by any of the conventional routes. However, as specified above, given the small size of their particles, they can be administered intravenously in the form of an aqueous suspension and are therefore compatible with the vascular microcirculation.

For obvious reasons, the amounts of derivatives according to the invention which can be used may vary significantly depending on the method of use and the route selected for their administration.

On the other hand, for topical administration, and most particularly for the treatment of melanomas, it may be envisioned to formulate at least one complex and/or nanoparticle in accordance with the present invention in a proportion of from 1% to 20% by weight, or even more, relative to the total weight of the pharmaceutical formulation under consideration.

It is also possible to coadminister ac least one complex and/or nanoparticle in accordance with the present invention with at least one other active material that is also capable of being beneficial with regard to the pathological condition under consideration.

By way of representation of these active materials that may be combined with the complex and/or nanoparticle in accordance with the present invention, mention may in particular be made of other anticancer or cytostatic molecules or macromolecules (for example, platinum salts, anthracyclines, mitotic spindle poisons, topoisomerase inhibitors, kinase inhibitors or metalloprotease inhibitors), anti-inflammatory agents of corticosteroid type (for example, dexamethasone) or non-corticosteroid type, or else molecules with immunoadjuvant activity (for example, antibodies with anticancer activity). In particular, by way of preferred combination, mention may be made of the combination of at least one complex and/or at least one nanoparticle according to the present invention with at least one compound chosen from cisplatin, carboplatin, tamoxifen, epirubicin, leuprolide, bicalutamide, goserelin implant, irinotecan, gemcitabine and sargramostim, or pharmaceutically acceptable salts thereof. These salts can be prepared with pharmaceutically acceptable acids, i.e. acids compatible in particular in terms of toxicity, for pharmaceutical use.

Combination with hyperthermia used in certain chemotherapies can be envisioned.

The complexes and/or nanoparticles in accordance with the present invention can also be combined with surgical therapies and/or with radiation for the treatment of cancer.

The examples which follow illustrate the present invention without, however, being limited thereto.

The infrared spectra are obtained by measurement on a pure solid or liquid using a Fourier spectrometer (Broker Vector® 22 Fourier Transform spectrometer). Only the significant absorptions are noted.

The optical rotations were measured using a Perkin-Elmer® 241 polarimeter, at a wavelength of 589 nm.

The ¹H and ¹³C NMR spectra were recorded using a Bruker AC® 200P spectrometer (at 200 MHz and 50 MHz, respectively, for ¹H and ¹³C) or a Bruker Avance® 300 spectrometer (at 300 MHz and 75 MHz, respectively, for ¹H and ¹³C).

The mass spectra were recorded using a Bruker Esquire-LC® instrument.

The thin layer chromatography analysis was carried out on plates pre-coated with silica 60F₂₅₄ gel (layer of 0.25 mm).

The column chromatography was carried out on silica 60 gel (Merck, 230-400 mesh ASTM).

All the reactions using compounds sensitive to air or to water were carried out under a hood.

### EXAMPLE 1

### a) Functionalization of squalene (obtaining N-[2-(pyridin-2-yldithio)ethyl]-1,1′,2-trisnorsqualenamide)

The functionalization of squalene can be represented schematically according to scheme 1 below.

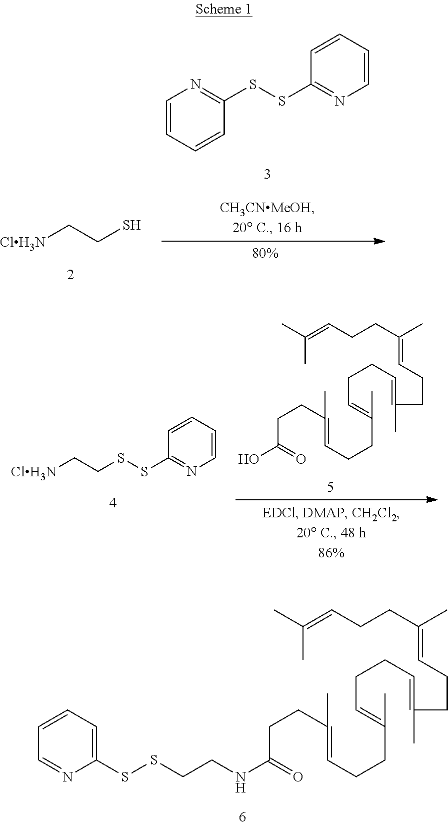

As indicated in scheme 1 above, in order to carry out the functionalization of squalene, the thioethanolamine 2 is activated with the 2,2′-dithiobispyridine 3, according to methods well known to those skilled in the art and in particular by analogy with the protocol described in Elbright et al., Biochemistry 1992, 31, 10664-10670, so as to obtain the 2-(pyridin-2-yldithio)ethanamine hydrochloride 4.

The derivative 4 is then condensed with the 1,1′,2-trisnorsqualenic acid 5 by reacting 1-ethyl-3-(3-dimethylaminopropyl)carbodiimide hydrochloride (EDCl) so as to give the N-[2-(pyridin-2-yldithio)ethyl]-1,1′,2-trisnorsqualenamide 6.

In particular, 226 mg (1 mmol) of 2-(pyridin-2-yldithio)ethanamine 4, 200 mg of triethylamine (2 mmol) and 236 mg of EDCl (1.5 mmol) are added to a solution of 1,1′,2-trisnorsqualenic acid 5 (400 mg, 1 mmol) in anhydrous dichloromethane (5 ml) and under an inert atmosphere. The mixture is stirred at ambient temperature for 48 h and then taken up in 10 ml of water and extracted with dichloromethane (3×20 ml). The organic phases are combined, washed with a saturated aqueous solution of NaCl, dried over MgSO₄, and concentrated under reduced pressure. The residue is purified by silica gel chromatography, elution being carried out with a 3:1 cyclohexane/ethyl acetate mixture.

490 mg of amide 6 are recovered in the form of a colorless oil:

IR (film) ν=3288, 2912, 2851, 1648 (CONH), 1637 (C═C, weak), 1575, 1543, 1446 cm⁻¹;

¹H NMR (CDCl₃, 300 MHz) δ=8.49 (d, J=4.8 Hz, 1H), 7.62 (td, J=7.6, 1.8 Hz, 1H), 7.52 (dd, J=8.1, 0.9 Hz, 1H), 7.13 (ddd, J=5.0, 6.0, 0.9 Hz, 1H), 7.03 (m, 1H, NHCO), 5.18-5.09 (m, 5H), 3.53 (q, J=5.6 Hz, 2H, CH₂NCO), 2.90 (t, J=5.6 Hz, 2H, CH₂SS), 2.15-1.95 (m, 20H), 1.67 (s, 3H), 1.63 (s, 3H), 1.59 (s, 12H);

¹³C NMR (50 MHz, CDCl₃) δ=172.8 (CO), 159.2 (N═C—S), 149.2 (N═CH), 136.9 (CH), 135.0 (C), 134.8 (2 C), 133.5 (C), 131.2 (C), 125.2 (CH), 124.3 (2 CH), 124.2 (2 CH), 1212 (CH), 120.9 (CH), 39.7 (CH₂), 39.6 (2 CH₂), 38.8 (CH₂), 37.1 (CH₂), 35.4 (CH₂), 35.3 (CH₂), 23.2 (2 CH₂), 26.7 (CH₂), 26.6 (CH₂). 25.6 (CH₃), 17.6 (CH₃), 16.0 (2 C₃), 15.8 (2 CH₃);

MS (+ESI, MeOH): m/z (%): 569 (69) [M+H]⁺, 591 (100) [M+Na]⁺.

b) Functionalization of the RET/PTC1 siRNA (SEQ ID No.: 1)

The RET/PTC1 siRNA (SEQ ID No.: 1) is obtained by synthesis of the oligonucleotide sequence, ordered from the company EUROGENTEC (Seraing, Belgium).

Such a synthesis is carried out according to the conventional methods well known to those skilled in the art.

The RET/PTC1 siRNA is functionalized with a thiol function, in particular represented by the 3-mercaptopropyl radical, according to the methods well known to those skilled in the art, as indicated above.

### EXAMPLE 2

### Conjugation of the RET/PTC1 siRNA (SEQ ID No: 1) with N-[2-(pyridin-2-yldithio)ethyl]-1,1′,2-trisnorsqualenamide

The conjugation of the RET/PTC1 siRNA with N-[2-(pyridin-2-yldithio)ethyl]-1,1′,2-trisnorsqualenamide can be illustrated by scheme 2 which follows:

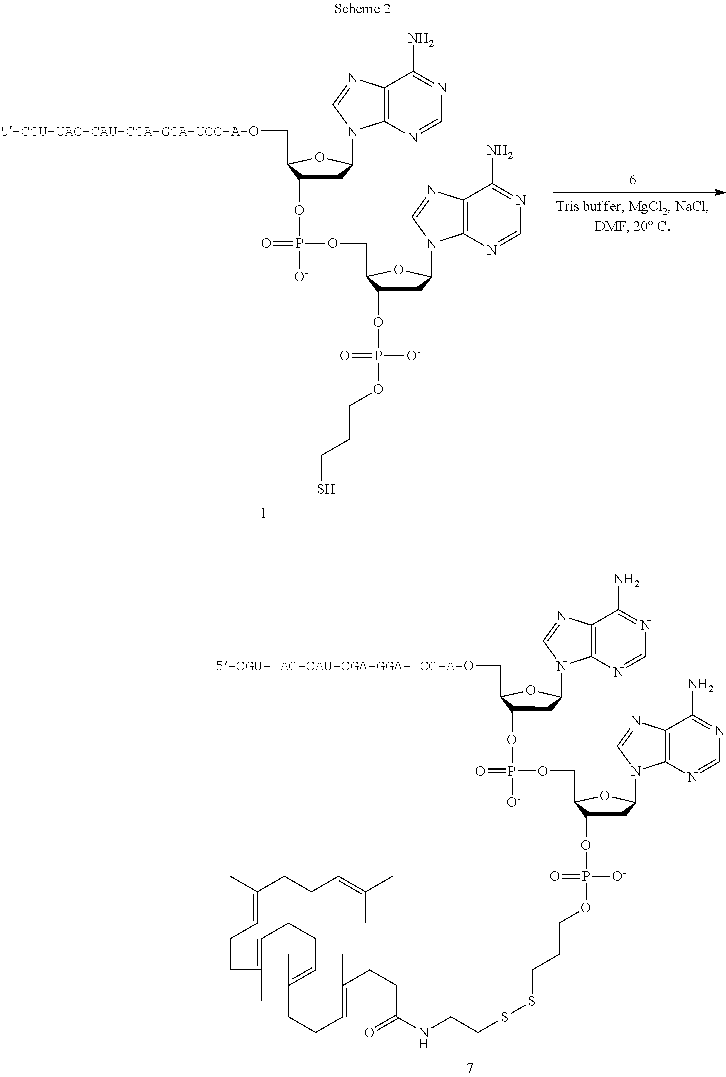

As indicated in scheme 2 above, the conjugation of the RET/PTC1 siRNA (SEQ ID No: 1) 1 with the N-[2-(pyridin-2-yldithio)ethyl]-1,1′,2-trisnorsqualenamide 6 is carried out by incubation at ambient temperature, in aqueous dimethylformamide (DMF), in a buffered medium. The excess compound 6 is then eliminated and the residue obtained is washed with an organic solvent, and then purified by HPLC.

In particular, 633 μl of a 100 mM ammonium acetate buffer solution and 33 μl of a 150 mM solution of the pirydyl disulfide derivative 6 as prepared in example 1a (2.8 mg, 5 μM) in freshly distilled DMF are added sequentially to a 0.15 mM solution of siRNA 1 in MilliQ water (333 μl, 0.34 mg, 0.050 μM). The mixture is incubated for 10 min at 20 C. and then concentrated under reduced pressure (0.5 Torr, 20° C.). The residue 7, corresponding to the RET/PTC1 siRNA/amido 1,1′,2-trisnorsqualene crude complex, is washed with ethyl ether (3×2 ml) and concentrated under vacuum.

The residue 7 is taken up in water and freeze-dried so as to give 340 μg of a white solid.

MS (−ESI, CH₃CN, Et₃N): m/z (%): 7254 (55) [M−H]⁻.

### EXAMPLE 3

### Preparation of the Nanoparticles (Purification in Water of the Complex Obtained According to Example 2)

500 μl of milliQ® water are added to the freeze-dried siRNA conjugated to squalene (siRNA-SQ), and then the mixture is centrifuged at 12 000 rpm (4° C., 10 min).

The centrifugation pellet is then dissolved in 500 μl of ethanol. The ethanolic solution is then added dropwise to MilliQ® water containing 5% dextrose with magnetic stirring, which is maintained for 5 min. The nanoparticle suspension thus formed is then transferred into a round-bottomed flask in order to evaporate off the ethanol in a Rotavapor. The volume is then adjusted to 1 ml with water (on a balance).

The size of the nanoparticles is measured by light scattering using the Nanosizer, and is found to be equal to 355 nm.

1. A complex formed by at least one molecule of nucleic acid comprising from 10 to 40 nucleotides, covalently coupled to at least one hydrocarbon-based compound, that is at least a C₁₈ is hydrocarbon-based compound, having a squalene structure or an analog thereof.

2. The complex as claimed claim 1, in which the hydrocarbon-based compound, that is at least a C₁₈ is hydrocarbon-based compound, of squalene structure or analog thereof is represented by the radical of formula (I) which follows: 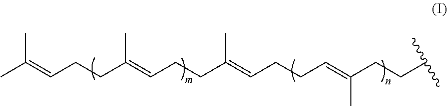
in which:
m=1, 2, 3, 4 or 5;
n=0, 1, 2, 3, 4 or 5; and 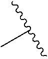
represents the bond to the rest of the complex with the nucleic acid.

3. The complex as claimed in claim 1, in which the hydrocarbon-based compound, that is at least a C₁₈ hydrocarbon-based compound, of squalene structure is 1,1′,2-trisnorsqualenic acid.

4. The complex as claimed in claim 1, in which the molecule of nucleic acid comprising from 10 to 40 nucleotides is an RNA molecule.

5. The complex as claimed in claim 1, in which the RNA molecule is an siRNA molecule.

6. The complex as claimed in claim 5, in which the siRNA is the RET/PTC1 siRNA.

7. The complex as claimed in claim 6, characterized in that the RET/PTC1 siRNA has a nucleotide sequence as follows: 5′-CGUUACCAUCGAGGAUCCAdAdA-3′ (SEQ ID No: 1).

8. The complex as claimed in any claim 1, in which the covalent coupling is a covalent bond of ester, ether, thioether disulfide, phosphate or amide type.

9. The complex as claimed in claim 1, represented by general formula (II) which follows: 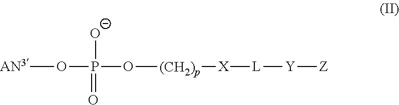
AN represents a molecule of nucleic acid comprising from 10 to 40 nucleotides, the 3′ end of which is linked to the rest of the complex,
X represents a covalent linkage between the two entities,
L represents a linker arm,
Y represents a covalent linkage,
p represents 0, 1, 2, 3 or 4,
Z represents a squalenoyl radical or derivative thereof, of formula (I) 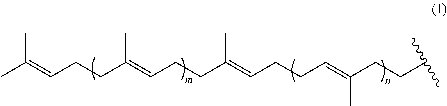
in which:
m=1, 2, 3, 4 or 5:
n=0, 1, 2, 3, 4 or 5; and 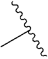
represents the bond to the rest of the complex with the nucleic acid.

10. The complex as claimed in claim 9, in which X is a disulfide covalent linkage and Z represents a radical of formula (I) in which m represents 1 and n represents 2.

11. Nanoparticles of a complex as described in claim 1.

12. The nanoparticles as claimed in claim 11, the mean size of which ranges from 30 to 500 nm.

13. A method for preparing nanoparticles as claimed in claim 11, characterized in that it comprises at least:
the dispersion of a complex formed by at least one molecule of nucleic acid comprising from 10 to 40 nucleotides, covalently coupled to at least one hydrocarbon-based compound that is at least a C₁₈ hydrocarbon-based compound having a squalene structure or an analog thereof, in at least one organic solvent, at a concentration that is sufficient to obtain, when the resulting mixture is added, with stirring, to an aqueous phase, the instantaneous formation of nanoparticles of said complex in suspension in said aqueous phase, and
where appropriate, the isolation of said nanoparticles.

14. A method for the treatment and/or prevention of cancers comprising at least a step of administering the nanoparticles as claimed in claim 11.

15. A pharmaceutical composition comprising at least one complex as described in claim 1 and/or nanoparticles of the complex, in combination with at least one acceptable pharmaceutical vehicle.

16. The complex as claimed in claim 8, in which the covalent coupling is a covalent bond of disulfide type.

17. The complex as claimed in claim 9, in which Y represents a linkage of ester, ether, thioether, disulfide, phosphate or amide type.

18. The complex as claimed in claim 9, in which X is a disulfide covalent linkage and Z represents a radical of formula (I) in which m represents 1 and n represents 2.

19. The nanoparticles as claimed in claim 12, the mean size of which ranges from 50 to 250 nm.

20. The nanoparticles as claimed in claim 12, the mean size of which ranges from 100 to 400 nm.

21. A method for the treatment and/or prevention of endocrine cancers comprising at least a step of administering the nanoparticles as claimed in claim 11.

22. A method for the treatment and/or prevention of papillary thyroid carcinoma comprising at least a step of administering the nanoparticles as claimed in claim 11.
